(19) United States
(12) Patent Application Publication
(10) Pub. No.: US 2024/0183851 A1
(43) Pub. Date: Jun. 6, 2024

(54) METHOD FOR PRODUCING ARTIFICIAL ANTIBODY, ARTIFICIAL ANTIBODY, AND BIOLOGICAL MATERIAL DETECTION SENSOR

(71) Applicant: SEIKO EPSON CORPORATION, TOKYO (JP)

(72) Inventors: Chikara KOJIMA, Matsumoto-shi (JP); Masamitsu MOCHIZUKI, CHINO-SHI (JP); Shinji TERAO, FUJIMI-MACHI (JP); Keita KUBO, Matsumoto-shi (JP); Kazuki Sano, MATSUMOTO-SHI (JP); Kiyoaki MURAI, MATSUMOTO-SHI (JP); Masahiko KOBAYASHI, CHINO-SHI (JP)

(21) Appl. No.: 18/530,270

(22) Filed: Dec. 6, 2023

(30) Foreign Application Priority Data
Dec. 6, 2022 (JP) 2022-195093

(51) Int. Cl. G01N 33/551

(52) U.S. Cl. CPC G01N 33/551

(57) ABSTRACT

A method for producing an artificial antibody includes: an alignment step of supplying a dispersion containing monomers and molding patterns implemented by a biological material or a replica of the biological material to a guide, and aligning the molding patterns by the guide; a polymer layer forming step of forming a polymer layer by polymerizing the monomers in a state where the molding patterns are aligned; and a removal step of removing the molding patterns from the polymer layer and obtaining an artificial antibody including the polymer layer having a template with a three-dimensional structure complementary to a three-dimensional structure of the molding pattern.

The present application is based on, and claims priority from JP Application Serial Number 2022-195093, filed Dec. 6, 2022, the disclosure of which is hereby incorporated by reference herein in its entirety.

### BACKGROUND

### 1. Technical Field

The present disclosure relates to a method for producing an artificial antibody, an artificial antibody, and a biological material detection sensor.

### 2. Related Art

In the fields of food hygiene, medical diagnosis, environmental monitoring, and the like, it is expected to implement a sensor that quickly and easily detects a biological material such as bacteria.

For example, WO 2014/156584A discloses a sensor including a detection unit including a detection electrode and a polymer layer having a template with a three-dimensional structure complementary to a three-dimensional structure of a microorganism to be detected, and a crystal for detecting a mass change or the like in the detection unit. The template of the polymer layer has a three-dimensional structure complementary to the three-dimensional structure of the microorganism, and thus the template selectively traps a microorganism having the three-dimensional structure, but does not trap a microorganism that does not have the three-dimensional structure. Therefore, a target microorganism can be detected with high sensitivity based on a capturing state in the template.

In WO 2014/156584A, a polymer layer is formed using an actual microorganism. Specifically, WO 2014/156584A discloses a method for forming a polymer layer including a step of forming a polymer layer such that a microorganism is incorporated, and a step of destroying the incorporated microorganism. Accordingly, a template having a three-dimensional structure complementary to a three-dimensional structure of an actual microorganism is formed in the polymer layer. The polymer layer having such a template is also called an artificial antibody.

In the artificial antibody described in WO 2014/156584A, it is necessary to use an actual microorganism for the formation of the artificial antibody. However, when an artificial antibody is produced using an actual microorganism, it is difficult to control a presence density of the microorganism to be constant. Therefore, there is a problem that the artificial antibody described in WO 2014/156584A has a large individual difference in arrangement density of the templates. When the individual difference in the arrangement density of the templates is large, the microorganism detection accuracy of the sensor is lowered.

### SUMMARY

A method for producing an artificial antibody according to an application example of the present disclosure includes: an alignment step of supplying a dispersion containing monomers and molding patterns implemented by a biological material or a replica of the biological material to a guide, and aligning the molding patterns by the guide; a polymer layer forming step of forming a polymer layer by polymerizing the monomers in a state where the molding patterns are aligned; and a removal step of removing the molding patterns from the polymer layer and obtaining an artificial antibody including the polymer layer having a template with a three-dimensional structure complementary to a three-dimensional structure of the molding pattern.

An artificial antibody according to an application example of the present disclosure includes: a polymer layer having a template with a three-dimensional structure complementary to a three-dimensional structure of a biological material; and a guide that supports the polymer layer and has a recessed portion provided at a position corresponding to the template.

A biological material detection sensor according to an application example of the present disclosure includes: the artificial antibody according to the application example of the present disclosure; and a resonance device whose resonance frequency changes with trapping of a biological material to the template.

### BRIEF DESCRIPTION OF THE DRAWINGS

FIG. 1 is a schematic diagram showing a bacterium as an example of a biological material, and is a cross-sectional view of an artificial antibody according to an embodiment.

FIG. 2 is an enlarged view of a portion A1 and a portion A3 in FIG. 1.

FIG. 3 is a flowchart showing a method for producing the artificial antibody in FIG. 1.

FIG. 4 is a cross-sectional view illustrating the production method shown in FIG. 3, and is a schematic diagram showing the bacterium as an example of a biological material and a cross-sectional view of a replica of the bacterium.

FIG. 5 is a cross-sectional view illustrating the production method shown in FIG. 3, and is an enlarged view of the portion A1 and a portion A2 in FIG. 4.

FIG. 6 is a cross-sectional view illustrating the production method shown in FIG. 3, and shows a state where a dispersion containing replicas and monomers is supplied to a guide.

FIG. 7 is a cross-sectional view illustrating the production method shown in FIG. 3, and shows a state where the replica is accommodated in a recessed portion of the guide.

FIG. 8 is a cross-sectional view illustrating the production method shown in FIG. 3, and shows a state where a polymer layer is formed.

FIG. 9 is a cross-sectional view illustrating the production method shown in FIG. 3, and is an enlarged view of a portion A4 in FIG. 8.

FIG. 10 is a cross-sectional view illustrating the production method shown in FIG. 3, and shows a state where the replica is removed from the polymer layer.

FIG. 11 is a cross-sectional view illustrating the production method shown in FIG. 3, and is an enlarged view of a portion A5 in FIG. 10.

FIG. 12 is a cross-sectional view illustrating the production method shown in FIG. 3, and shows a state where the polymer layer is heated and expanded.

FIG. 13 is a cross-sectional view illustrating the production method shown in FIG. 3, and shows a state where the replica is attracted by a magnetic attraction force.

FIG. 14 is a cross-sectional view schematically showing a biological material detection sensor according to the embodiment.

FIG. 15 is a modification of a resonance device shown in FIG. 14.

FIG. 16 is a modification of the resonance device shown in FIG. 14.

### DESCRIPTION OF EMBODIMENTS

Hereinafter, a method for producing an artificial antibody, an artificial antibody, and a biological material detection sensor of the present disclosure will be described in detail based on embodiments illustrated in the accompanying drawings.

### 1. Artificial Antibody

First, an artificial antibody according to an embodiment will be described.

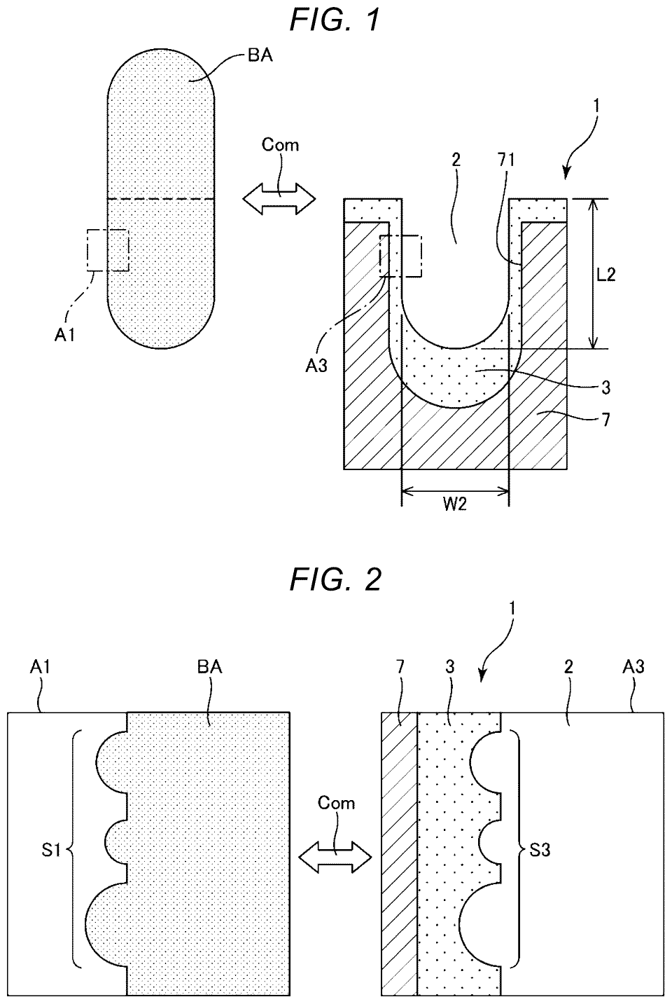

FIG. 1 is a schematic diagram showing a bacterium BA as an example of a biological material, and is a cross-sectional view of an artificial antibody 1 according to the embodiment. FIG. 2 is an enlarged view of a portion A1 and a portion A3 in FIG. 1.

The bacterium BA shown in FIG. 1 is an example of a biological material, and has a specific three-dimensional structure S1 as shown in FIG. 2 on a surface thereof. The three-dimensional structure S1 is derived from, for example, a specific molecular structure of the bacterium BA, and varies depending on the type of biological materials. On the other hand, the artificial antibody 1 shown in FIG. 1 includes a polymer layer 3 having a template 2 with a complementary structure S3 complementary to the three-dimensional structure S1 of the bacterium BA, and has a property of selectively trapping the bacterium BA. Therefore, a biosensor or the like that detects the bacteria BA with high sensitivity can be implemented using the artificial antibody 1.

Arrows Com shown in FIGS. 1 and 2 indicate that the three-dimensional structure S1 of the bacterium BA and the complementary structure S3 of the template 2 are complementary to each other. In the present specification, the term “complementary” means that at least a part of the three-dimensional structure S1 of the bacterium BA and the complementary structure S3 of the template 2 are combined with each other in shape without gaps, or means a relation that the two are combined with each other in shape without gaps by enlarging or reducing either one of them. The shapes to be combined allow deviation in the shapes due to production errors (gaps due to production errors). When either one of them is enlarged or reduced, an enlargement ratio and a reduction ratio are as small as, for example, about 5% or less with respect to an original size. Accordingly, it can be said that the template 2 is a model obtained by transferring the bacterium BA in a substantially original size.

A part to be transferred to the template 2 may be the entire bacterium BA or any part thereof. The template 2 shown in FIG. 1 is obtained by transferring a part of the rod-shaped bacterium BA in a longitudinal direction. In addition, the biological material is not limited to a bacterium, and may be, for example, a microorganism other than a bacterium, such as a virus, a fungus, and a protozoan, a cell, or an exosome.

Specific examples of the bacterium include Escherichia coli, Pseudomonas aeruginosa, Staphylococcus aureus, and Bacillus subtilis. Examples of the virus include a hepatitis A virus, an adenovirus, a rotavirus, and a norovirus. Examples of the fungus include Candida. Examples of the protozoan include Cryptosporidium.

The artificial antibody 1 shown in FIGS. 1 and 2 includes the polymer layer 3 having the template 2, and a guide 7 supporting the polymer layer 3 and having a recessed portion 71 provided at a position corresponding to the template 2.

When the longest axis that can be taken by the template 2 is defined as a major axis, a length L2 of the major axis varies depending on the type of biological materials. The length L2 is, for example, 0.3 μm or more and 10 μm or less in the case of a bacterium. When an axis perpendicular to the major axis is defined as a minor axis, a length W2 of the minor axis is, for example, 0.1 μm or more and 5 μm or less in the case of a bacterium.

The polymer layer 3 is formed by polymerizing the monomers. The monomers are not particularly limited, and examples thereof include pyrrole, aniline, thiophene, and derivatives thereof. For example, when pyrrole is used as the monomer, the polymer layer 3 becomes a polypyrrole layer.

The guide 7 supports the polymer layer 3 and guides a position of the template 2 when the template 2 is to be formed in the polymer layer 3. The guide 7 has the recessed portion 71, and a probability that the template 2 is formed at a position corresponding to the recessed portion 71 is increased. Therefore, when the guide 7 has the recessed portion 71, the artificial antibody 1 in which the templates 2 are arranged at an intended arrangement density can be implemented. Accordingly, the artificial antibody 1 having a small individual difference in the arrangement density of the templates 2 and the artificial antibody 1 in which the templates 2 are arranged at a higher density can be obtained.

The arrangement density of the templates 2 in the polymer layer 3 is preferably 1×10⁶ [number/cm²] or more, and more preferably 1×10⁸ [number/cm²] or more. The artificial antibody 1 by which a biosensor or the like with a particularly high detection sensitivity for the bacterium BA can be implemented can be obtained by implementing such an arrangement density. An arrangement density of the recessed portions 71 is considered to be the same as the arrangement density of the templates 2. Therefore, it is preferable that the arrangement density of the recessed portions 71 also satisfies the above-described range.

The arrangement density of the templates 2 is calculated from, for example, the number of templates 2 present in an image obtained by imaging the polymer layer 3 with a scanning electron microscope (SEM) and an area of the image.

As will be described later, the recessed portion 71 regulates a position of a molding pattern for forming the template 2. Accordingly, a size and a depth of the recessed portion 71 are appropriately set in accordance with a size of the molding pattern so as to regulate the position. Accordingly, a length of a major axis of the recessed portion 71 can be set within the same range as that of the length L2 of the major axis of the above-described template 2. A length of a minor axis of the recessed portion 71 can also be set within the same range as that of the length W2 of the minor axis of the above-described template 2.

A constituent material of the guide 7 is not particularly limited, and examples thereof include an organic material such as a resin, and an inorganic material such as a metal, a ceramic, a glass, and a crystal. Among them, a crystalline material is preferably used, and quartz crystal, single crystalline silicon, or polycrystalline silicon is more preferably used. These materials are a material which can be finely processed by using micro electro mechanical systems (MEMS) processing technique, that is, photolithography and etching, and thus these materials are useful as the constituent material of the guide 7. These materials are also used as a material of a vibrator element of a resonator. Therefore, as will be described later, the guide 7 itself can also be used as a vibrator element.

The resin has a small specific gravity, and thus the guide 7 that can enhance a detection sensitivity of a biological material can be implemented, for example, when the artificial antibody 1 is applied to a biosensor or the like.

Examples of the resin include a thermoplastic resin, a thermosetting resin, and a photocurable resin. When the constituent material of the guide 7 includes a resin, the cost of the guide 7 can be easily reduced. The guide 7 having high dimensional accuracy can be obtained by utilizing the formability of the resin. The resin has a small specific gravity, and thus the guide 7 that can enhance a detection sensitivity of a biological material can be implemented, for example, when the artificial antibody 1 is applied to a biosensor or the like.

Examples of the thermoplastic resin include polyvinyl acetates, polyvinyl alcohols, polyvinyl butyrals, polystyrenes, an acrylonitrile butadiene styrene (ABS) resin, a methacrylic resin, a noryl resin, polyurethanes, an ionomer resin, cellulose-based plastic, polyethylenes, polypropylenes, polyamides, polycarbonates, polyacetals, polyphenylene sulfides, polyvinylidene chlorides, polyesters, and a fluororesin. One kind of them may be used alone, or two or more kinds thereof may be used in combination.

Examples the of thermosetting resin and the photocurable resin include polyimides, an epoxy resin, a phenol resin, a urea resin, a melamine resin, a silicone resin, polyamide imides, benzocyclobutenes, benzoxazines, and a cyanate resin. One kind of them may be used alone, or two or more kinds thereof may be used in combination.

Examples of the metals include a simple substance of a metallic element such as Fe, Co, Ni, Cu, Ti, Al, Mg, Ag, Au, Pt, Mo, W, Nb, and Ta, and an alloy containing the metallic element as a main component.

Examples of the ceramic include oxide-based ceramics such as silicon oxides, magnesium oxides, calcium oxides, aluminum oxides, titanium oxides, zirconium oxides, boron oxides, and yttrium oxides, and non-oxide-based ceramics such as silicon nitrides, aluminum nitrides, boron nitrides, titanium nitrides, silicon carbides, boron carbides, titanium carbides, and tungsten carbides.

Examples of the glass include quartz glass and borosilicate glass.

### 2. Method for Producing Artificial Antibody

Next, a method for producing an artificial antibody according to an embodiment will be described. In the following description, a method for producing the artificial antibody 1 according to the above-described embodiment will be described as an example.

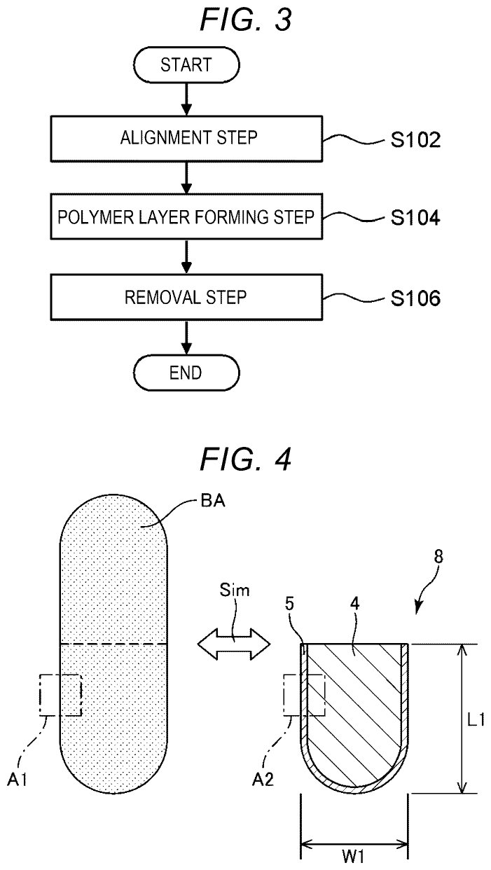

FIG. 3 is a flowchart showing a method for producing the artificial antibody 1 in FIG. 1. FIGS. 4 to 13 are cross-sectional views illustrating the production method shown in FIG. 3. In FIGS. 4 to 13, the same components as those in FIGS. 1 and 2 are denoted by the same reference signs.

The method for producing the artificial antibody 1 shown in FIG. 3 includes an alignment step S102, a polymer layer forming step S104, and a removal step S106.

### 2.1. Alignment Step

In the alignment step S102, a dispersion containing monomers and molding patterns implemented by the bacterium BA or a replica 8 of the bacterium BA is supplied to the guide 7, and the molding patterns are aligned by the guide 7. The molding pattern is a pattern for forming the template 2, and may be either of the actual bacterium BA and the replica 8 thereof. In the present embodiment, the replica 8 of the bacterium BA is used. The replica 8 has a similar structure S2 similar to the three-dimensional structure S1 of the bacterium BA.

The replica 8 can be reused once or a plurality of times even after being used for producing the artificial antibody 1. Therefore, the artificial antibody 1 can be produced more efficiently by using the replica 8 as compared with the case of producing the artificial antibody 1 by using the actual bacterium BA.

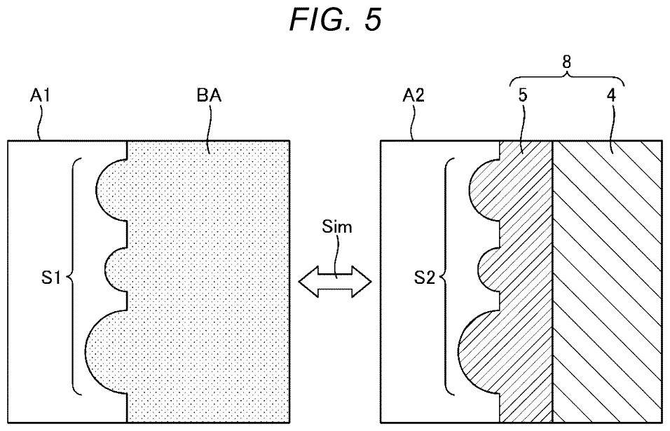

FIG. 4 is a schematic diagram showing the bacterium BA as an example of a biological material, and is a cross-sectional view of the replica 8 of the bacterium BA. FIG. 5 is an enlarged view of the portion A1 and a portion A2 in FIG. 4.

Arrows Sim shown in FIGS. 4 and 5 indicate that the three-dimensional structure S1 of the bacterium BA and the similar structure S2 of the replica 8 are similar to each other. In the present specification, the term “similar” means that at least a part of the three-dimensional structure S1 of the bacterium BA and the similar structure S2 of the replica 8 have the same shape, or means a relation that the two have the same shape by enlarging or reducing either one of them. The same shape allows deviation in the shape due to a production error. When either one of them is enlarged or reduced, an enlargement ratio and a reduction ratio are as small as, for example, about 5% or less with respect to an original size. Accordingly, it can be said that the replica 8 is a model obtained by imitating the bacterium BA in a substantially original size.

A part imitated by the replica 8 may be the entire bacterium BA or any part thereof. The replica 8 shown in FIG. 4 is obtained by imitating a part of the rod-shaped bacterium BA in the longitudinal direction. In addition, the biological material is not limited to a bacterium, and may be, for example, a microorganism other than a bacterium, such as a virus, a fungus, and a protozoan, a cell, or an exosome.

The replica 8 shown in FIGS. 4 and 5 has a core-shell structure including a body portion 4 and a coating film 5 for coating a surface of the body portion 4. Therefore, the similar structure S2 is a part obtained by forming a part of the coating film 5 into a three-dimensional shape. The coating film 5 is preferably made of an inorganic material. The inorganic material is superior to an organic material in mechanical properties such as mechanical strength, rigidity, and wear resistance, and chemical properties such as corrosion resistance and heat resistance. Therefore, when the coating film 5 is made of an inorganic material, that is, the surface is made of an inorganic material, the replica 8 in which the similar structure S2 is stably maintained can be obtained.

When the longest axis that can be taken by the replica 8 is defined as a major axis, a length L1 of the major axis varies depending on the type of the biological materials. The length L1 is, for example, 0.3 μm or more and 10 μm or less in the case of a bacterium. When an axis perpendicular to the major axis is defined as a minor axis, a length W1 of the minor axis is, for example, 0.1 μm or more and 5 μm or less in the case of a bacterium.

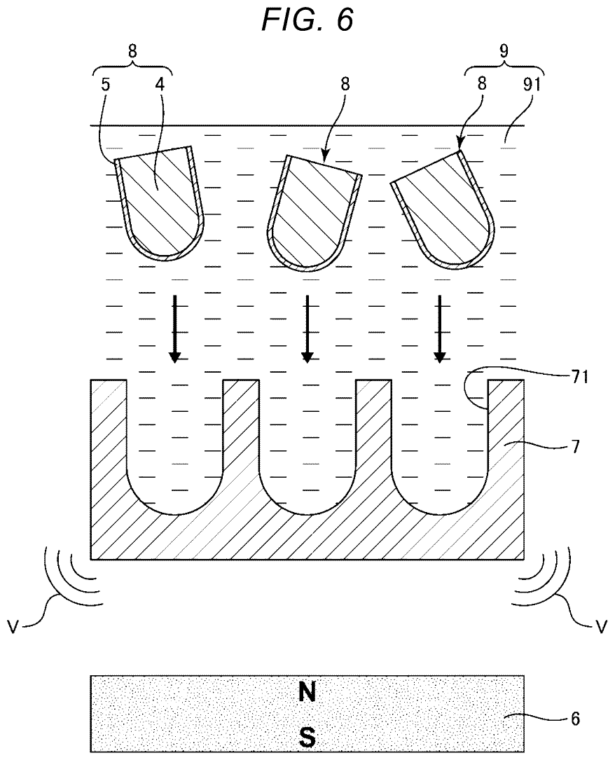

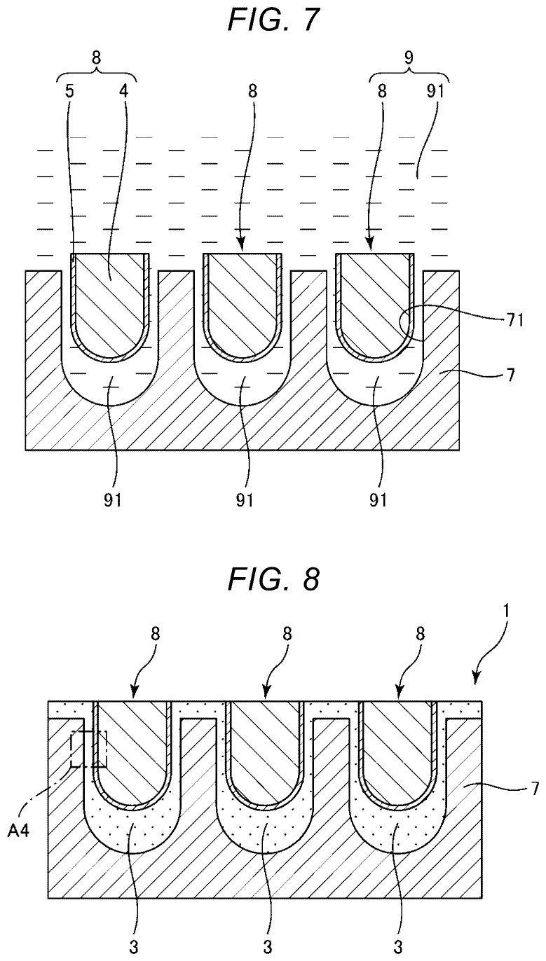

In the alignment step S102, a dispersion 9 containing the replicas 8 and monomers 91 is supplied to the guide 7 as shown in FIG. 6. Accordingly, the replica 8 repeatedly comes into contact with the guide 7 while moving in the dispersion. Then, the replica 8 enters the recessed portion 71 with a predetermined probability. As a result, as shown in FIG. 7, the replicas 8 are respectively accommodated in the recessed portions 71. At this time, the monomers 91 exist between the recessed portion 71 and the replica 8.

As described above, it is preferable that the recessed portions 71 accommodate a part or all of the replicas 8. Accordingly, the behavior of the replicas 8 dispersed in the dispersion 9 is easily controlled, and the replicas 8 are easily aligned.

The guide 7 may be configured such that the replicas 8 are aligned in a shape other than the recessed portion 71, such as a protruding portion, a protruding stripe, and a liquid-repellent portion.

The guide 7 is formed in, for example, a plate shape as shown in FIG. 6. The recessed portion 71 is opened in one main surface of the guide 7. In this case, when the dispersion 9 is supplied to the surface on which the recessed portion 71 is opened, a contact probability between the replica 8 and the recessed portion 71 is easily increased.

The alignment step S102 may include an operation of applying vibration V to the dispersion 9. This operation increases the probability that the replica 8 moves so as to be accommodated in the recessed portion 71. Accordingly, the arrangement density of the templates 2 can be freely controlled. The vibration V is applied by, for example, a vibration generator that applies vibration to the guide 7 or an ultrasonic generator that irradiates the dispersion 9 with ultrasonic waves.

The alignment step S102 may include an operation of sucking the dispersion 9. Specifically, a suction hole may be formed in a bottom portion of the recessed portion 71, and a suction pressure may be applied from the suction hole by a vacuum pump or the like. This operation increases the probability that the replica 8 moves so as to be accommodated in the recessed portion 71. Accordingly, the arrangement density of the templates 2 can be freely controlled.

Examples of a constituent material of the body portion 4 include organic materials such as resins, and inorganic materials such as metals, metal-based compounds, non-metals, and non-metal-based compounds. A composite material obtained by combining an organic material and an inorganic material may be used.

Examples of the metals include a simple substance of a metallic element such as Fe, Co, Ni, Cu, Ti, Al, Mg, Ag, Au, Pt, Mo, W, Nb, and Ta, and an alloy containing the metallic element as a main component. Examples of the metal-based compound include metal oxides, metal carbides, metal nitrides, metal chlorides, metal sulfides, metal carbonates, and metal hydroxides.

Examples of the non-metals include Si, B, and C. Examples of the non-metal-based compounds include non-metal oxides, non-metal carbides, and non-metal nitrides.

The constituent material of the body portion 4 may be a composite material obtained by combining two or more kinds selected from the above-described inorganic materials, or may be a composite material obtained by combining at least one kind selected from the above-described inorganic materials and another inorganic material.

Examples of the resin include a thermoplastic resin, a thermosetting resin, and a photocurable resin. In particular, when a thermosetting resin or a photocurable resin is used, the rigidity and heat resistance of the body portion 4 can be increased as compared with the case of using a thermoplastic resin. In the case of the photocurable resin, the resin is polymerized by light irradiation, and thus a volume change in the resin due to curing is small, and the shape and size of the original template can be maintained with higher accuracy for the replica 8.

Examples of the thermoplastic resin include polyvinyl acetates, polyvinyl alcohols, polyvinyl butyrals, polystyrenes, an acrylonitrile butadiene styrene (ABS) resin, a methacrylic resin, a noryl resin, polyurethanes, an ionomer resin, cellulose-based plastic, polyethylenes, polypropylenes, polyamides, polycarbonates, polyacetals, polyphenylene sulfides, polyvinylidene chlorides, polyesters, and a fluororesin. One kind of them may be used alone, or two or more kinds thereof may be used in combination.

Examples of the thermosetting resin and the photocurable resin include polyimides, an epoxy resin, a phenol resin, a urea resin, a melamine resin, a silicone resin, polyamide imides, benzocyclobutenes, benzoxazines, and a cyanate resin. One kind of them may be used alone, or two or more kinds thereof may be used in combination.

The coating film 5 is made of an inorganic material as described above. Examples of the inorganic material include oxides, nitrides, carbides, sulfides, and fluorides. Among them, the inorganic material is preferably an oxide or a nitride. The oxide and the nitride are particularly excellent in mechanical properties and chemical properties among the inorganic materials. Therefore, the coating film 5 capable of particularly stably maintaining the similar structure S2 can be obtained.

Examples of the oxide include silicon oxides, titanium oxides, aluminum oxides, zirconium oxides, hafnium oxides, niobium oxides, zinc oxides, tin oxides, yttrium oxides, magnesium oxides, calcium oxides, and boron oxides.

Among them, silicon oxides, titanium oxides, and aluminum oxides are preferably used. These oxides can form the dense coating film 5, and are particularly good in mechanical properties and chemical properties. The silicon oxides are oxides represented by a composition formula SiOₓ (0<x≤2), and are preferably SiO₂. The titanium oxides are oxides represented by a composition formula TiOₓ (0<x≤2), and are preferably TiO₂. The aluminum oxides are oxides represented by a composition formula AlOₓ (0<x≤2), and are preferably Al₂O₃.

Examples of the nitrides include gallium nitrides, silicon nitrides, aluminum nitrides, boron nitrides, and titanium nitrides.

The constituent material of the coating film 5 may be a composite material obtained by combining two or more kinds selected from the above-described inorganic materials, or a composite material obtained by combining at least one kind selected from the above-described inorganic materials and another inorganic material. Examples of the composite material include an Al₂O₃—SiO₂ composite material.

An average thickness of the coating film 5 is not particularly limited, and is preferably 1 nm or more and 500 nm or less, more preferably 3 nm or more and 100 nm or less, and still more preferably 5 nm or more and 50 nm or less. When the average thickness of the coating film 5 is within the above range, a thickness sufficient for forming the similar structure S2 can be ensured, and an adverse effect caused by the coating film 5 being too thick can be prevented. That is, when the average thickness of the coating film 5 is less than the lower limit, there is a possibility that the similar structure S2 having various protrusion heights cannot be sufficiently formed. On the other hand, when the average thickness of the coating film 5 exceeds the upper limit, it takes more time to form the coating film 5, and thus the production efficiency of the replica 8 may decrease.

The average thickness of the coating film 5 is determined by observing a cross section of the replica 8 with a scanning electron microscope (SEM) or a scanning transmission electron microscope (STEM), measuring film thicknesses of 10 or more positions freely selected, and calculating an average value thereof.

The replica 8 may contain a magnetic material. In this case, the alignment step S102 may include an operation of applying a magnetic field to the dispersion 9 for alignment. The magnetic material has a property of being aligned along a direction of the magnetic field, so that the replicas 8 can be efficiently aligned by this operation. Examples of a method for applying a magnetic field include a method for bringing a magnetic field generation source 6 close to the guide 7 as shown in FIG. 6. Examples of the magnetic field generation source 6 include a permanent magnet and an electromagnet.

The magnetic material may be a paramagnetic material, and is preferably a ferromagnetic material. The ferromagnetic material has a high permeability, and thus a large magnetic attraction force can be generated in response to the applied magnetic field.

The ferromagnetic material may be a hard magnetic material, and is preferably a soft magnetic material. The soft magnetic material has a high permeability and a low coercivity. Therefore, a large magnetic attraction force is generated in a state where the magnetic field is applied, and on the other hand, the magnetic attraction force can be greatly reduced when the application of the magnetic field is stopped. Accordingly, the alignment of the replicas 8 in the alignment step S102 and the removal of the replicas 8 in the removal step S106 to be described later can be efficiently performed by using on/off of the application of the magnetic field. The coercivity of the soft magnetic material is preferably 800 A/m or less, and more preferably 400 A/m or less.

Examples of the soft magnetic material include metal-based soft magnetic materials such as an Fe-based alloy, a Ni-based alloy, and a Co-based alloy, and oxide-based soft magnetic materials such as a spinel ferrite, a magnetoplumbite ferrite, and a garnet ferrite which all contain Fe₃O₄ as a main component. Among them, an oxide-based soft magnetic material is preferably used. The oxide-based soft magnetic materials are useful as a magnetic material contained in the replica 8 because particles of a nm-size can be produced at low cost.

On the other hand, when the molding pattern is the bacterium BA, an electric field may be applied to the dispersion 9. The bacteria BA are generally negatively charged. Therefore, the bacteria BA can be migrated to a guide 7 side by appropriately setting a direction of the electric field.

### 2.2. Polymer Layer Forming Step

In the polymer layer forming step S104, the monomers are polymerized in a state where the replicas 8 are aligned, and the polymer layer 3 is formed. With the above-described alignment step S102, the replica 8 is accommodated in each recessed portion 71 of the guide 7 as shown in FIG. 7. The monomers 91 exist between the recessed portion 71 and the replica 8.

In the polymer layer forming step S104, a polymerization reaction is caused in the monomers 91 in the dispersion 9. Accordingly, as shown in FIG. 8, the polymer layer 3 can be obtained.

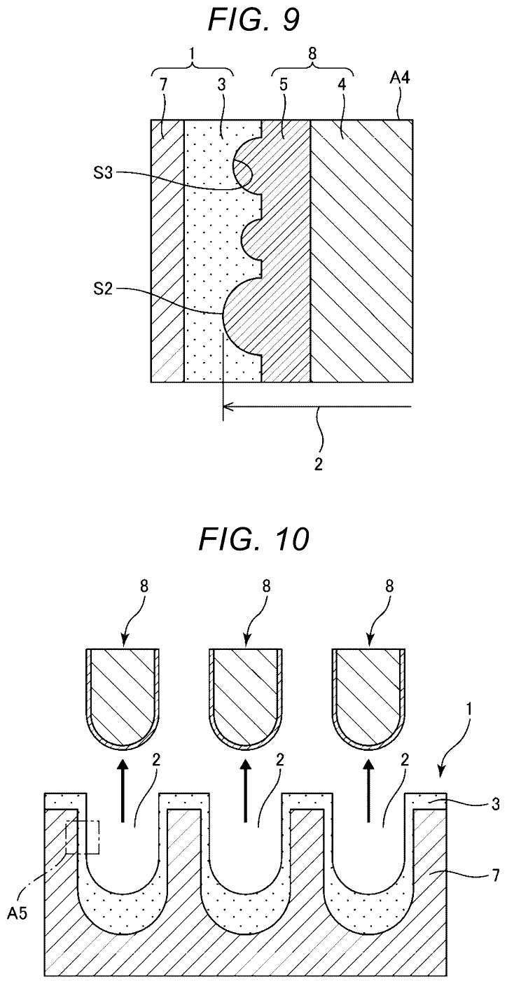

FIG. 9 is an enlarged view of a portion A4 in FIG. 8. As shown in FIG. 9, the similar structure S2 of the replica 8 is transferred to the polymer layer 3. As a result, the polymer layer 3 having the template 2 with the complementary structure S3 shown in FIG. 9 can be obtained.

The monomers are not particularly limited, and examples thereof include pyrrole, aniline, thiophene, and derivatives thereof. For example, when pyrrole is used as the monomers, the polymer layer 3 becomes a polypyrrole layer. In this case, examples of the polymerization reaction include an electrolytic polymerization reaction, and the polymerization reaction in this step is not limited thereto.

### 2.3. Removal Step

In the removal step S106, the replicas 8 are removed from the polymer layer 3 as shown in FIG. 10. Accordingly, the artificial antibody 1 including the polymer layer 3 having the template 2 with the complementary structure S3 (complementary three-dimensional structure) can be obtained. FIG. 11 is an enlarged view of a portion A5 in FIG. 10. The complementary structure S3 shown in FIG. 11 is a structure complementary to the similar structure S2 (three-dimensional structure of the molding pattern) of the replica 8.

Examples of a method for removing the replica 8 include a method for performing an operation of pulling out the replica 8 from the polymer layer 3. At this time, in order to minimize the influence on the template 2, the following operation is preferably used in combination.

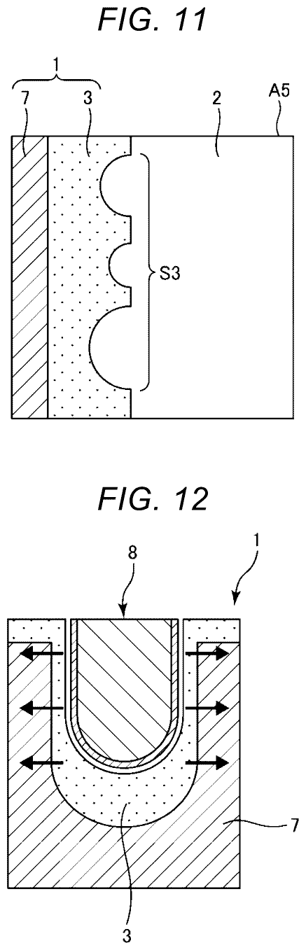

First, an operation in which the polymer layer 3 is heated to expand can be exemplified. In this operation, the polymer layer 3 is expanded by heating. Accordingly, a gap can be generated between the polymer layer 3 and the replica 8 as shown in FIG. 12. As a result, the replica 8 can be smoothly separated and removed. The heating temperature is not particularly limited as long as it is a temperature at which the polymer layer 3 is not thermally denatured, and is, for example, 40° C. or higher and 100° C. or lower. When the body portion 4 of the replica 8 is made of an inorganic material, the replica 8 can be separated even by heating at a lower temperature because of a large difference in thermal expansion from the polymer layer 3.

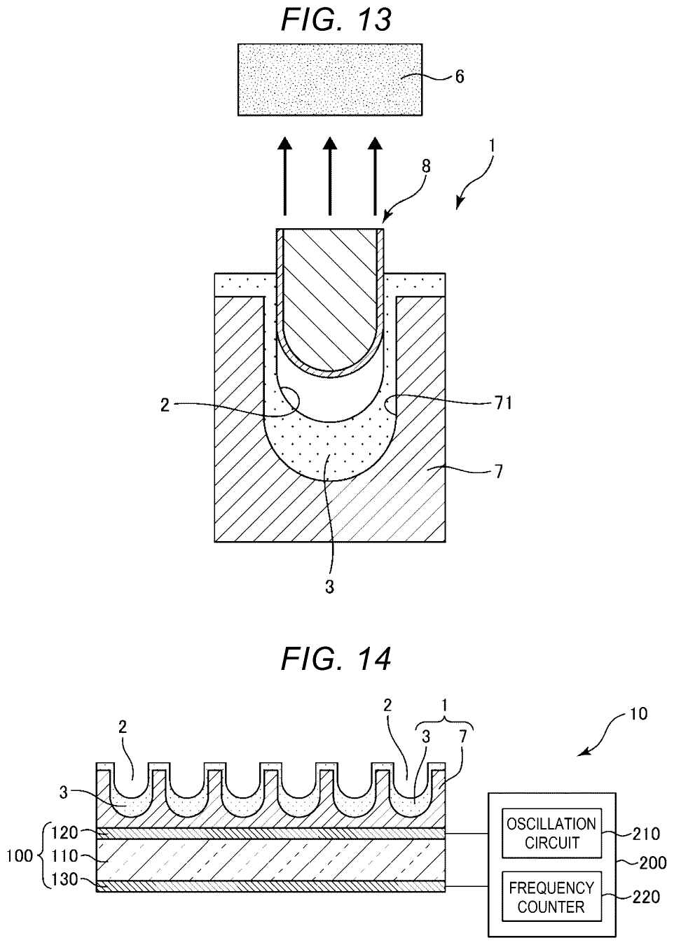

When the replica 8 contains a magnetic material and a magnetic field is applied to the replica 8, a magnetic attraction force is generated in the replica 8. Specifically, as shown in FIG. 13, when the magnetic field generation source 6 is brought close to the replica 8 from an opening side of the recessed portion 71 of the guide 7, a magnetic attraction force toward the magnetic field generation source 6 is generated for the replica 8. Accordingly, the replica 8 can be more easily removed. Examples of the magnetic field generation source 6 include a permanent magnet and an electromagnet. Among them, the electromagnet is preferably used from the viewpoint that the collected replica 8 can be easily separated from the magnetic field generation source 6.

According to the production method as described above, the replicas 8 are aligned using the guide 7 and the polymer layer 3 is formed in this state, and thus the replicas 8 can be arranged at the intended arrangement density. Accordingly, the artificial antibody 1 having a small individual difference in the arrangement density of the templates 2 can be produced. The arrangement density of the replicas 8 can be increased, and thus the artificial antibody 1 having a high arrangement density of the templates 2 can be produced. The artificial antibody 1 contributes to implementation of a biosensor or the like that detects the bacterium BA with high sensitivity.

In particular, by using the replica 8 as the molding pattern, the polymer layer 3 can be produced without using the actual bacterium BA. In addition, the replica 8 can be repeatedly used as a molding pattern replacing the bacterium BA. Therefore, the polymer layer 3 can be efficiently produced, and the cost of the artificial antibody 1 can be easily reduced. Even in the case of the bacterium BA that is difficult to obtain, the artificial antibody 1 can be stably produced in accordance with the demand.

### 3. Biological Material Detection Sensor

Next, a biological material detection sensor according to the embodiment will be described.

FIG. 14 is a cross-sectional view schematically showing a biological material detection sensor 10 according to the embodiment.

The biological material detection sensor 10 shown in FIG. 14 includes the artificial antibody 1, a resonance device 100, and a control device 200. A resonance frequency of the resonance device 100 changes as the bacterium BA (biological material) is trapped in the template 2. The control device 200 causes the resonance device 100 to oscillate and detects the bacterium BA based on a change in the resonance frequency of the resonance device 100.

As described above, the artificial antibody 1 shown in FIG. 14 includes the polymer layer 3 having the templates 2 and the guide 7 supporting the polymer layer 3. The resonance device 100 shown in FIG. 14 supports a surface of the guide 7 opposite to a surface thereof supporting the polymer layer 3.

The resonance device 100 includes a resonator. The resonator is an element that generates a constant frequency by using a piezoelectric phenomenon of a piezoelectric body 110. Examples of the type of the resonator include a crystal resonator, a silicon resonator, and a ceramic resonator. A fundamental resonance frequency of the resonator is maintained constant with high accuracy, but the resonance frequency of the resonator changes when a mass change occurs in the resonator.

As the resonator used for the resonance device 100, a crystal resonator is preferably used, and an AT cut quartz crystal resonator is more preferably used. In the crystal resonator, energy loss is small, and as a result, vibration can be stably continued. The AT cut quartz crystal resonator has a fundamental resonance frequency in the MHz band, and can detect a mass change more accurately than a resonator in the kHz band.

The resonance device 100 shown in FIG. 14 includes the piezoelectric body 110 and electrodes 120 and 130 laminated in a manner of sandwiching the piezoelectric body 110. The artificial antibody 1 is in contact with the piezoelectric body 110 via the electrode 120. The piezoelectric body 110 shown in FIG. 14 is a thin piece having a substantially constant thickness. In the case of the crystal resonator, the thin piece is a crystal. The thickness of the piezoelectric body 110 affects the detection sensitivity of the mass change and the drive force necessary for the oscillation, and thus the thickness is appropriately selected in accordance with a constituent material of the artificial antibody 1 and the like. As an example, the thickness of the piezoelectric body 110 is preferably 10 μm or more and 200 μm or less.

The control device 200 includes an oscillation circuit 210 and a frequency counter 220. The oscillation circuit 210 applies an AC voltage to the resonance device 100 to cause the resonance device 100 to oscillate. The frequency counter 220 measures a resonance frequency based on signals output from the oscillation circuit 210 and calculates a change from the fundamental resonance frequency. The control device 200 detects the mass change based on a change width of the resonance frequency. The biological material detection sensor 10 quantitatively detects the bacterium BA against the artificial antibody 1 from the detected mass change. In the artificial antibody 1, the individual difference in the arrangement density of the templates 2 is small, and the arrangement density can be easily increased. Therefore, the biological material detection sensor 10 including the artificial antibody 1 can detect the bacterium BA with higher accuracy and higher sensitivity.

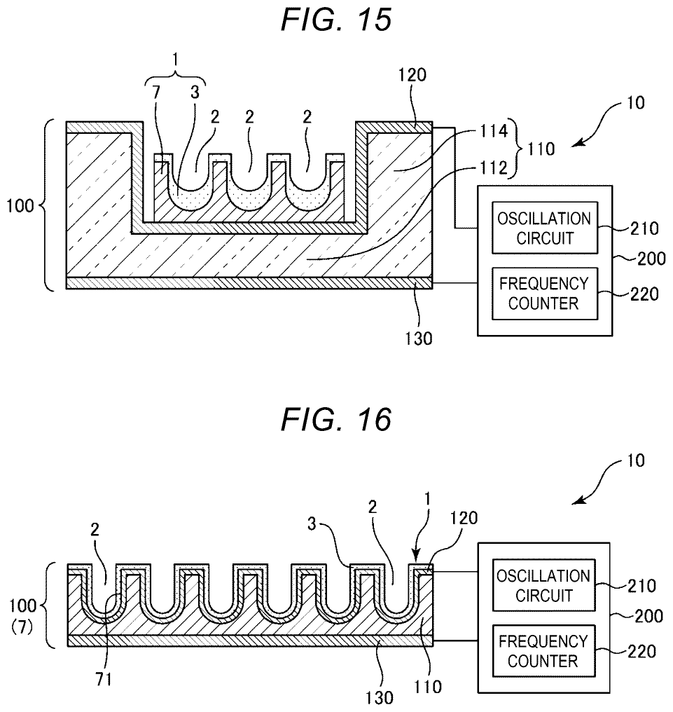

The resonance device 100 shown in FIG. 15 is a modification of the resonance device 100 shown in FIG. 14.

In the piezoelectric body 110 shown in FIG. 15, a thin film portion 112 located on an inner side and a thick film portion 114 located on an outer side thereof have different thicknesses. The artificial antibody 1 is supported by the thin film portion 112.

According to such a configuration, both high detection sensitivity in the thin film portion 112 and large drive force in the thick film portion 114 can be achieved. That is, even when the number of bacteria BA trapped by the artificial antibody 1 is small, the trapping state of the bacteria BA can be accurately detected in the thin film portion 112, and the biological material detection sensor 10 with high sensitivity can be implemented. The bacteria BA are generally supplied with a dispersion medium. Therefore, the resonance device 100 is required to have a drive force by which oscillation can be continued even under the influence of the viscosity of the dispersion medium. A large drive force is obtained in the thick film portion 114, so that oscillation of the thin film portion 112 can be continued even in a state where the dispersion medium is in contact with the artificial antibody 1.

The thickness of the thin film portion 112 is preferably 3 μm or more and 50 μm or less, and more preferably 5 μm or more and 20 μm or less. The thickness of the thick film portion 114 may be larger than that of the thin film portion 112, and is preferably 80 μm or more and 500 μm or less, and more preferably 150 μm or more and 250 μm or less.

The resonance device 100 shown in FIG. 16 is a modification of the resonance device 100 shown in FIG. 14.

The resonance device 100 shown in FIG. 16 also has the function of the guide 7. In other words, the guide 7 shown in FIG. 16 also functions as the resonance device 100. Accordingly, the resonance device 100 shown in FIG. 16 has the recessed portion 71 and supports the polymer layer 3 having the template 2 corresponding to the position of the recessed portion 71.

According to such a configuration, the influence of the mass of the artificial antibody 1 on the oscillation of the resonance device 100 can be minimized. That is, since the resonance device 100 also has the function of the guide 7, the weight of the artificial antibody 1 is reduced by the weight of the guide 7. As a result, the resonance device 100 that is more likely to oscillate even under the influence of the dispersion medium can be obtained.

The configurations of the resonance device 100 shown in FIGS. 14, 15, and 16 are examples, and may be appropriately combined. For example, in FIG. 15, the artificial antibody 1 is disposed only in a vicinity of a central portion of the resonance device 100, and the ends are reinforced. Alternatively, the ends may not be reinforced. Accordingly, the resonance device has higher sensitivity than the configuration in FIG. 14, and is easier to produce than the configuration in FIG. 15.

The configuration of the resonance device 100 shown in FIG. 15 and the configuration of the resonance device 100 shown in FIG. 16 may be combined. That is, the function of the guide 7 may be created by treating a surface of the thin film portion 112 shown in FIG. 15. Accordingly, the resonance device 100 that more easily oscillates can be obtained. As a result, the bacterium BA can be quantitatively detected with high sensitivity, and the biological material detection sensor 10 that is easy to handle can be implemented.

### 4. Effects of Embodiment

As described above, the method for producing the artificial antibody 1 according to the embodiment includes the alignment step S102, the polymer layer forming step S104, and the removal step S106. In the alignment step S102, the dispersion 9 containing the replicas 8 (the biological material or the molding pattern implemented by the replicas) and the monomers 91 is supplied to the guide 7, and the replicas 8 are aligned by the guide 7. In the polymer layer forming step S104, the monomers 91 are polymerized in a state where the replicas 8 are aligned, and the polymer layer 3 is formed. In the removal step S106, the replicas 8 are removed from the polymer layer 3 to obtain the artificial antibody 1 including the polymer layer 3 having the template 2 with the complementary structure S3 (complementary three-dimensional structure) complementary to the similar structure S2 (the three-dimensional structure of the molding pattern) of the replicas 8.

According to such a configuration, the replicas 8 are aligned using the guide 7 and the polymer layer 3 is formed in this state, and thus the replicas 8 can be arranged at the intended arrangement density. Accordingly, the artificial antibody 1 having a small individual difference in the arrangement density of the templates 2 can be produced. The artificial antibody 1 contributes to implementation of the biological material detection sensor 10 or the like that detects the bacterium BA with high accuracy. The arrangement density of the replicas 8 can be increased, and thus the artificial antibody 1 having a high arrangement density of the templates 2 can be produced. The artificial antibody 1 contributes to implementation of the biological material detection sensor 10 or the like that detects the bacterium BA with high sensitivity.

The guide 7 preferably has the recessed portion 71 for accommodating the replica 8 (molding pattern). Accordingly, the behavior of the replicas 8 dispersed in the dispersion 9 is easily controlled, and the replicas 8 are easily aligned.

The alignment step S102 may include an operation of applying vibration V to the dispersion 9. Accordingly, the probability that the replicas 8 move so as to be accommodated in the recessed portion 71 is increased. As a result, the arrangement density of the template 2 can be freely controlled.

It is preferable that the molding pattern is implemented by the replica 8 of the bacterium BA (biological material). Accordingly, the polymer layer 3 can be produced without using the actual bacterium BA. In addition, the replica 8 can be repeatedly used as a molding pattern replacing the bacterium BA. Therefore, the polymer layer 3 can be efficiently produced, and the cost of the artificial antibody 1 can be easily reduced. Even in the case of the bacterium BA that is difficult to obtain, the artificial antibody 1 can be stably produced in accordance with the demand.

The replica 8 may contain a magnetic material. In this case, the alignment step S102 may include an operation of applying a magnetic field to the dispersion 9 for alignment. Accordingly, the replicas 8 can be efficiently aligned.

In this case, the removal step S106 may include an operation of applying a magnetic field to the replica 8 for attraction. Accordingly, the magnetic attraction force is generated for the replica 8, and the replica 8 can be more easily removed.

The replica 8 preferably has a surface made of an inorganic material. Accordingly, the replica 8 in which the similar structure S2 is stably maintained can be obtained.

The removal step S106 may include an operation of heating and expanding the polymer layer 3. Accordingly, a gap can be generated between the polymer layer 3 and the replica 8. As a result, the replica 8 can be smoothly separated and removed.

The artificial antibody 1 according to the embodiment includes the polymer layer 3 and the guide 7. The polymer layer 3 has the template 2 with the complementary structure S3 (three-dimensional structure) complementary to the three-dimensional structure S1 of the bacterium BA (biological material). The guide 7 supports the polymer layer 3 and has the recessed portion 71 provided at a position corresponding to the template 2.

According to such a configuration, the artificial antibody 1 having a small individual difference in the arrangement density of the templates 2 and the artificial antibody 1 in which the templates 2 are arranged at a higher density can be obtained. Accordingly, the biological material detection sensor 10 including the artificial antibody 1 can detect the bacterium BA with higher accuracy and higher sensitivity.

The guide 7 may include a crystal. The crystal includes the thin film portion 112 and the thick film portion 114 thicker than the thin film portion 112. The polymer layer 3 is provided at the thin film portion 112.

According to such a configuration, both high detection sensitivity in the thin film portion 112 and large drive force in the thick film portion 114 can be achieved. A large drive force is obtained in the thick film portion 114, so that oscillation of the thin film portion 112 can be continued even in a state where the dispersion medium is in contact with the artificial antibody 1.

The arrangement density of the templates 2 in the polymer layer 3 is preferably 1×10⁶ [number/cm²] or more. Accordingly, the artificial antibody 1 by which the biological material detection sensor 10 or the like having particularly high detection sensitivity of the bacterium BA (biological material) can be implemented can be obtained.

The biological material detection sensor 10 according to the embodiment includes the artificial antibody 1 and the resonance device 100. A resonance frequency of the resonance device 100 changes as the bacterium BA (biological material) is trapped in the template 2.

In the artificial antibody 1, an individual difference in the arrangement density of the templates 2 is small, and the arrangement density can be easily increased. Therefore, the biological material detection sensor 10 including the artificial antibody 1 can detect the bacterium BA with higher accuracy and higher sensitivity.

Although the method for producing an artificial antibody, the artificial antibody, and the biological material detection sensor of the present disclosure were described above based on the illustrated embodiments, the present disclosure is not limited thereto. For example, in the artificial antibody and the biological material detection sensor of the present disclosure, a configuration of each portion of the embodiment may be replaced with any configuration having the same function, and any other components may be added to the embodiment. The method for producing an artificial antibody of the present disclosure may be a method in which a step for any purpose is added to the embodiment.

What is claimed is:

1. A method for producing an artificial antibody, comprising:
an alignment step of supplying a dispersion containing monomers and molding patterns implemented by a biological material or a replica of the biological material to a guide, and aligning the molding patterns by the guide;
a polymer layer forming step of forming a polymer layer by polymerizing the monomers in a state where the molding patterns are aligned; and
a removal step of removing the molding patterns from the polymer layer and obtaining an artificial antibody including the polymer layer having a template with a three-dimensional structure complementary to a three-dimensional structure of the molding patterns.

2. The method for producing an artificial antibody according to claim 1, wherein
the guide has a recessed portion configured to accommodate the molding patterns.

3. The method for producing an artificial antibody according to claim 1, wherein
the alignment step includes an operation of applying vibration to the dispersion.

4. The method for producing an artificial antibody according to claim 1, wherein
the molding patterns are a replica of a biological material,
the replica contains a magnetic material, and
the alignment step includes an operation of applying a magnetic field to the dispersion for alignment.

5. The method for producing an artificial antibody according to claim 1, wherein
the molding patterns are a replica of a biological material,
the replica contains a magnetic material, and
the removal step includes an operation of applying a magnetic field to the replica for attraction.

6. The method for producing an artificial antibody according to claim 1, wherein
the molding patterns are a replica of a biological material, and
the replica has a surface made of an inorganic material.

7. The method for producing an artificial antibody according to claim 1, wherein
the removal step includes an operation of heating and expanding the polymer layer.

8. An artificial antibody comprising:
a polymer layer having a template with a three-dimensional structure complementary to a three-dimensional structure of a biological material; and
a guide configured to support the polymer layer and having a recessed portion provided at a position corresponding to the template.

9. The artificial antibody according to claim 8, wherein
the guide includes a crystal,
the crystal includes a thin film portion and a thick film portion thicker than the thin film portion, and
the polymer layer is provided at the thin film portion.

10. The artificial antibody according to claim 8, wherein an arrangement density of the template in the polymer layer is 1×10⁶ [number/cm²] or more.

11. A biological material detection sensor comprising:
the artificial antibody according to claim 8; and
a resonance device whose resonance frequency changes with trapping of a biological material to the template.
